(19) United States
(12) Patent Application Publication
(10) Pub. No.: US 2021/0181836 A1
(43) Pub. Date: Jun. 17, 2021

(54) INFORMATION PROCESSING APPARATUS, INFORMATION PROCESSING METHOD, AND PROGRAM

(71) Applicant: SONY CORPORATION, TOKYO (JP)

(72) Inventor: YUKI YAMAMOTO, CHIBA (JP)

(21) Appl. No.: 16/769,881

(22) PCT Filed: Sep. 26, 2018
(86) PCT No.: PCT/JP2018/035695

§ 371 Date: Jun. 4, 2020

(30) Foreign Application Priority Data
Dec. 15, 2017 (JP) 2017-240255

(51) Int. Cl. G06F 3/01; G06K 9/00; G06T 7/73

(52) U.S. Cl. CPC G06F 3/013; G06T 7/75; G06K 9/00208; G06K 9/00604

(57) ABSTRACT

[Problem] To realize line-of-sight estimation with high accuracy in accordance with an individual characteristic. [Solution] Provided is an information processing apparatus that includes an arithmetic processing unit that performs arithmetic processing related to estimation of a line of sight of a user by using an eyeball model. The arithmetic processing unit dynamically estimates an individual parameter related to the eyeball model for each of users, and the individual parameter includes information on a relative position of a structure constituting an eyeball in a three-dimensional space. Further provided is an information processing method for performing, by a processor, arithmetic processing related to estimation of a line of sight of a user by using an eyeball model. The performing of the arithmetic processing includes dynamically estimating an individual parameter related to the eyeball model for each of users, and the individual parameter includes information on a relative position of a structure constituting an eyeball in a three-dimensional space.

### FIELD

The present disclosure relates to an information processing apparatus, an information processing method, and a program.

### BACKGROUND

In recent years, a technology for estimating a user's line of sight and using the estimated line of sight for various kinds of operation has been in widespread use. Further, a number of technologies for improving accuracy of line-of-sight detection have been developed. For example, Patent Literature 1 discloses a technology for improving detection accuracy of a corneal reflection image on a cornea in line-of-sight estimation using a corneal reflection method.

### CITATION LIST

### Patent Literature

Patent Literature 1: Japanese Laid-open Patent Publication No. 2016-106668

### SUMMARY

### Technical Problem

Meanwhile, if line-of-sight estimation using an eyeball model, such as the corneal reflection method, is performed, it is important to use an eyeball model that closely resembles an eyeball structure unique to a user in order to obtain a line-of-sight estimation result with high accuracy. However, in the technology described in Patent Literature 1, the line-of-sight estimation is performed by using an eyeball model that is determined in advance, so that it may be difficult to deal with a new user in some cases.

Therefore, in the present disclosure, an information processing apparatus, an information processing method, and a program that are novel, modified, and capable of realizing line-of-sight estimation with high accuracy in accordance with an individual characteristic.

### Solution to Problem

According to the present disclosure, an information processing apparatus is provided that includes: an arithmetic processing unit that performs arithmetic processing related to estimation of a line of sight of a user by using an eyeball model, wherein the arithmetic processing unit dynamically estimates an individual parameter related to the eyeball model for each of users, and the individual parameter includes information on a relative position of a structure constituting an eyeball in a three-dimensional space.

Moreover, according to the present disclosure, an information processing method is provided that includes: performing, by a processor, arithmetic processing related to estimation of a line of sight of a user by using an eyeball model, wherein performing the arithmetic processing includes dynamically estimating an individual parameter related to the eyeball model for each of users, and the individual parameter includes information on a relative position of a structure constituting an eyeball in a three-dimensional space.

Moreover, according to the present disclosure, a program is provided that causes a computer to function as an information processing apparatus that includes: an arithmetic processing unit that performs arithmetic processing related to estimation of a line of sight of a user by using an eyeball model, wherein the arithmetic processing unit dynamically estimates an individual parameter related to the eyeball model for each of users, and the individual parameter includes information on a relative position of a structure constituting an eyeball in a three-dimensional space.

### Advantageous Effects of Invention

As described above, according to the present disclosure, it is possible to realize line-of-sight estimation with high accuracy in accordance with an individual characteristic.

Meanwhile, the effects described above are not limitative, and, with or in the place of the above effects, any of the effects described in this specification or other effects that can be recognized from this specification may be achieved.

### BRIEF DESCRIPTION OF DRAWINGS

FIG. 1 is a diagram for explaining a flow of line-of-sight estimation using a corneal reflection method.

FIG. 2 is a diagram for explaining factors to reduce line-of-sight estimation accuracy in the corneal reflection method.

FIG. 3 is a diagram illustrating an arrangement example of hardware in a case where an information processing apparatus according to one embodiment of the present disclosure is a wearable device.

FIG. 4 is a schematic side view illustrating a positional relationship between an eyeball of a user and the information processing apparatus in a case where the information processing apparatus according to the embodiment is worn on a head of the user.

FIG. 5 is a block diagram illustrating a functional configuration example of the information processing apparatus according to the embodiment.

FIG. 6 is a diagram illustrating an example of reduction of the line-of-sight estimation accuracy due to an individual difference of an eyeball structure.

FIG. 7 is a diagram for explaining minimization of error variance in a target coordinate system according to one embodiment of the present disclosure.

FIG. 8 is a diagram for explaining an effect of the line-of-sight estimation using an individual parameter that is estimated by a full search method according to the embodiment.

FIG. 9 is a diagram for explaining a calculation of a cornea-pupil distance d by a closed-form solution according to the embodiment.

FIG. 10 is a diagram illustrating a degree of influence of the factor to reduce the line-of-sight estimation accuracy when individual parameter estimation according to the embodiment and calibration are performed.

FIG. 11 is a flowchart illustrating a flow of a line-of-sight estimation process according to the embodiment.

FIG. 12 is a flowchart illustrating a flow of individual parameter estimation using a plurality of target points according to the embodiment.

FIG. 13 is a flowchart illustrating a flow of individual parameter estimation using the closed-form solution according to the embodiment.

FIG. 14 is a diagram illustrating a hardware configuration example according to one embodiment of the present disclosure.

### DESCRIPTION OF EMBODIMENTS

Preferred embodiments of the present disclosure will be described in detail below with reference to the accompanying drawings. In this specification and the drawings, structural elements that have substantially the same functions and configurations will be denoted by the same reference symbols, and repeated explanation of the structural elements will be omitted.

In addition, hereinafter, explanation will be given in the following order.

1. Embodiment
1.1. Overview
1.2. Arrangement example of hardware related to information processing apparatus 10
1.3. Functional configuration example of information processing apparatus 10
1.4. Details of line-of-sight estimation
1.5. Flow of processes

2. Hardware configuration example

3. Conclusion

### 1. Embodiment

<<1.1. Overview>>

One example of a flow of line-of-sight estimation using a corneal reflection method will be described below. The corneal reflection method (also referred to as a pupil-corneal reflection method) is a method of applying light from a light source to an eyeball of a user and detecting reflected light and a position of a pupil on a corneal surface, to thereby estimate a user's line-of-sight direction.

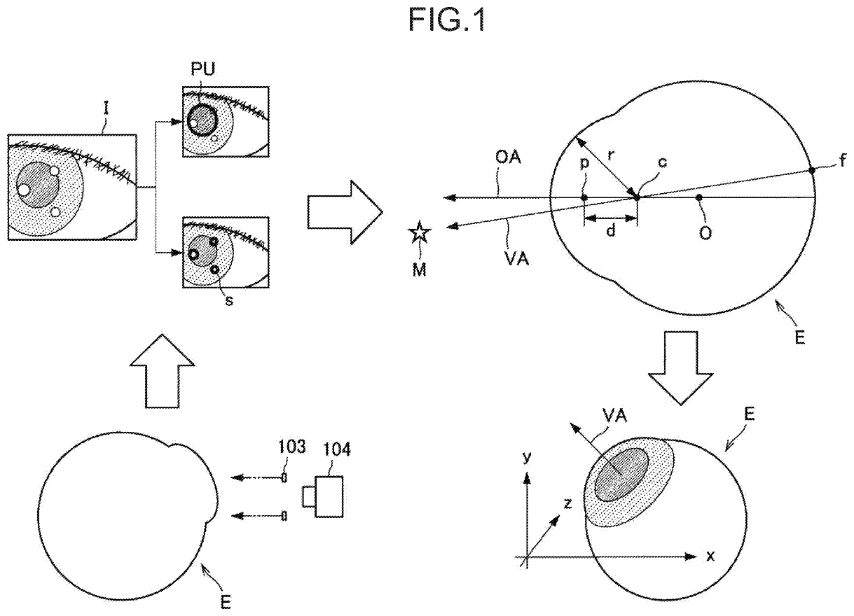

FIG. 1 is a diagram for explaining the flow of the line-of-sight estimation using the corneal reflection method. As illustrated in a lower left part in FIG. 1, an information processing apparatus that performs the line-of-sight estimation using the corneal reflection method first applies light from a light source 103 to an eyeball E of a user and causes an imaging unit 104 to capture an image including a corneal reflection image (also referred to as a Purkinje image or a bright point) on a corneal surface.

In an upper left part in FIG. 1, an eyeball image I that is obtained through the procedure as described above is illustrated. Subsequently, the information processing apparatus detects a pupil PU and a bright point s from the eyeball image I through image processing. In this case, the information processing apparatus may detect the pupil PU and the bright point s by using a statistical method, such as machine learning.

Then, the information processing apparatus calculates a line-of-sight vector of the user by using the detected pupil PU, the detected bright point s, and a three-dimensional eyeball model (hereinafter, also simply referred to as an eyeball model). An overview of a line-of-sight vector calculation using the eyeball model is illustrated on a right side in FIG. 1.

The information processing apparatus estimates a three-dimensional position of a corneal curvature center c that corresponds to a center of a cornea when the cornea is assumed as a spherical structure, on the basis of a position of the detected bright point and a position of the light source 103, for example. In this case, the information processing apparatus may obtain the three-dimensional position of the corneal curvature center c by using a corneal curvature radius r that is one of parameters (hereinafter, also referred to as eyeball parameters) related to the eyeball model.

Subsequently, the information processing apparatus estimates a three-dimensional position of a pupil center p, on the basis of the three-dimensional position of the corneal curvature center c and a cornea-pupil distance d that is one of the eyeball parameters. Meanwhile, the cornea-pupil distance d is an eyeball parameter that indicates a distance between the pupil center p and the corneal curvature center c.

Then, the information processing apparatus estimates an optical axis from the corneal curvature center c and the pupil center p that are estimated through the procedures as described above. For example, the information processing apparatus estimates a straight line connecting the corneal curvature center c and the pupil center p as the optical axis, and estimates a vector that extends from the corneal curvature center c through the pupil center p as an optical axis vector OA. In the corneal reflection method, the optical axis vector OA is detected as a line-of-sight direction of the user.

However, the optical axis vector OA estimated as described above and an actual line-of-sight direction of the user (a line-of-sight vector VA) deviate from each other A point of gaze (target point M) that is actually gazed at by the user is located on a visual axis connecting a central fovea f and the corneal curvature center c, and, in general, a difference (deviation) of about 4° to 8° occurs between the optical axis vector OA and the line-of-sight vector VA. Therefore, in the line-of-sight estimation using the corneal reflection method, it is common to perform calibration to correct deviation between the optical axis vector OA and the line-of-sight vector VA in order to improve accuracy of the line-of-sight estimation.

In the above explanation using FIG. 1, the example has been described in which the information processing apparatus uses the vector connecting the pupil center p and the corneal curvature center c as an estimation result, but the information processing apparatus may use a vector connecting the corneal curvature center c and an eyeball center O (rotation center) as the estimation result, for example.

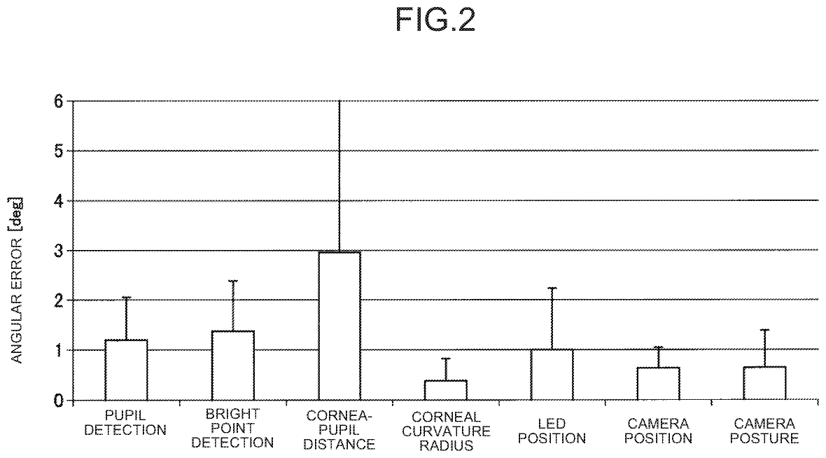

Further, in the line-of-sight estimation using the corneal reflection method, it is known that various factors lead to reduction of line-of-sight estimation accuracy. FIG. 2 is a diagram for explaining factors to reduce the line-of-sight estimation accuracy in the corneal reflection method. In FIG. 2, a horizontal axis represents the factors to reduce the line-of-sight estimation accuracy, and a vertical axis represents a magnitude of an angular error that occurs due to each of the factors.

With reference to FIG. 2, the factors for reduction are largely classified into three kinds. In other words, the factors include a detection error due to image processing including pupil detection and bright point detection, an error of the eyeball parameter including the cornea-pupil distance and the corneal curvature radius, and an error due to a hardware mounting position including an LED position, a camera position, and camera posture.

Further, with reference to FIG. 2, among the above-described two kinds of factors for reduction, it is indicated that the error of the eyeball parameter including the cornea-pupil distance has the highest degree of influence. The error due to the eyeball parameter as described above is caused by a difference between an eyeball structure of the user and an eyeball model used in the line-of-sight estimation. It is general that eyeball structures of humans vary among different individuals and the cornea-pupil distance and the corneal curvature radius vary for each of users. Therefore, for example, if the line-of-sight estimation is performed using an average eyeball model, a difference between an actual eyeball structure of a certain user may be largely different from the average eyeball model and the line-of-sight estimation accuracy may eventually be reduced.

A technical idea according to one embodiment of the present disclosure is conceived in view of the foregoing point and makes it possible to realize the line-of-sight estimation with high accuracy in accordance with an individual characteristic. Therefore, one of features of the information processing apparatus, the information processing method, and the program according to one embodiment of the present disclosure is to dynamically estimate an individual parameter related to an eyeball model for each of users. Further, the individual parameter as described above is an eyeball parameter that is unique to a user and that is related to the eyeball model, and may include information on a relative position of a structure of an eyeball in a three-dimensional space.

With the feature of the information processing apparatus, the information processing method, and the program according to one embodiment of the present disclosure as described above, it is possible to perform the line-of-sight estimation using a highly-accurate eyeball model for each of users, so that it is possible to improve accuracy of the line-of-sight estimation.

The above-described feature of the information processing apparatus, the information processing method, and the program according to the embodiment and effects achieved by the feature will be described in detail below.

<<1.2. Arrangement Example of Hardware Related to Information Processing Apparatus 10>>

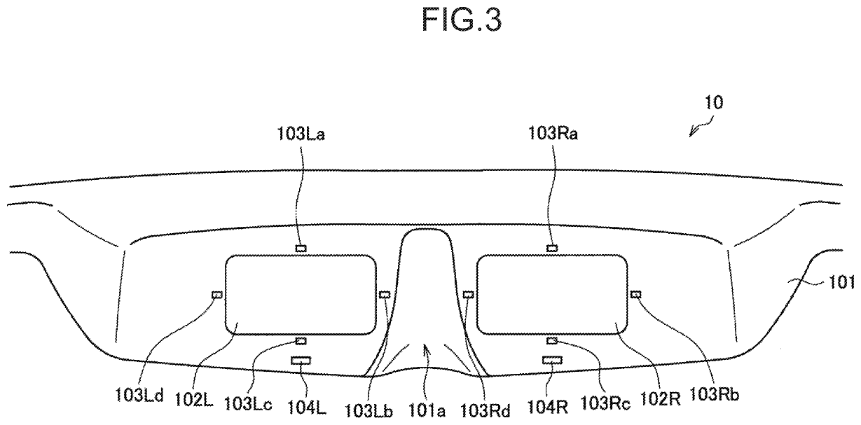

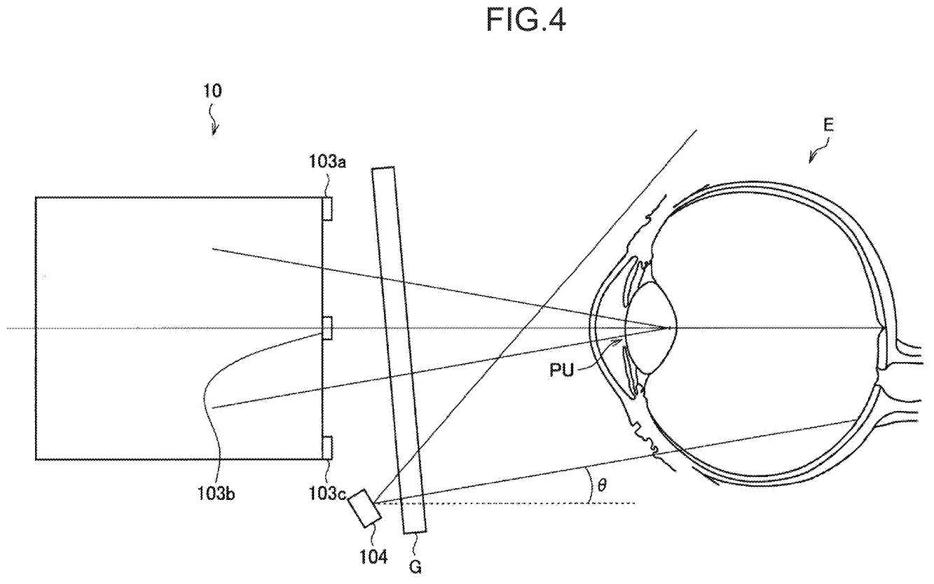

One example of arrangement of hardware of an information processing apparatus 10 according to the embodiment of the present disclosure will be described below. The information processing apparatus 10 according to the embodiment may be, for example, a head-mounted display to be worn on a head of a user or a glasses-type wearable device. FIG. 3 is a diagram illustrating a hardware arrangement example in a case where the information processing apparatus 10 according to the embodiment is a wearable device. Further, FIG. 4 is a schematic side view illustrating a positional relationship between an eyeball E of a user and the information processing apparatus 10 in a case where the information processing apparatus 10 is worn on a head of the user.

FIG. 3 illustrates a configuration of the information processing apparatus 10 when viewed from a side that faces eyes of the user. With reference to FIG. 3, the information processing apparatus 10 according to the embodiment includes displays 102R and 102L at positions corresponding to a right eye and a left eye of the user. As illustrated in FIG. 3, the displays 102R and 102L according to the embodiment may be formed in approximately rectangular shapes. Further, in a body 101, a recessed portion 101a at which a nose of the user is located may be formed between the displays 102R and 102L.

The displays 102R and 102L according to the embodiment may be, for example, liquid crystal displays, organic electroluminescence (EL) displays, or lenses on which information is displayed by a projection device.

Four light sources 103Ra to 103Rd are arranged at approximate centers of four sides around the display 102R. Similarly, for light sources 103La to 103Ld are arranged at approximate centers of four sides around the display 102L. The light sources 103Ra to 103Rd and 103La to 103d according to the embodiment may be infrared light emitting diodes (IR LEDs) that emit infrared light, for example. The light sources 103Ra to 103Rd and 103La to 103d emit infrared light to the opposing right eye and the opposing left eye of the user.

Meanwhile, the light sources 103Ra to 103Rd and 103La to 103d according to the embodiment need not always be the IR LEDs, but may be light sources that emit light with an appropriate wavelength capable of detecting the bright point.

Further, imaging units 104R and 104L that capture images of the eyeballs E of the user are arranged in the peripheries of the displays 102R and 102L. The imaging units 104R and 104L are arranged on lower sides of the displays 102R and 102L (below the light sources 103Rc and 103Lc) as illustrated in FIG. 3, for example.

Furthermore, as illustrated in FIG. 4, the imaging units 104R and 104L are arranged such that at least pupils PU of the eyeballs E to be captured are included in imaging ranges. For example, the imaging units 104R and 104L may be arranged at a predetermined elevation angle θ. The elevation angle θ may be, for example, about 30°.

Meanwhile, the information processing apparatus 10 is configured such that the displays 102R and 102L are separated from the eyeballs E of the user by a predetermined distance when the information processing apparatus is worn on the user. With this configuration, the user who has worn the information processing apparatus 10 is able to view display regions of the displays 102R and 102L in the user's visual field without feeling discomfort. In this case, it may be possible to determine the distance between each of the displays 102R and 102L and the eyeballs E of the user such that even when the user wears a glasses G, it is possible to wear the information processing apparatus 10 on the glasses G in an overlapping manner. The imaging units 104R and 104L are arranged such that the pupils PU of the eyeballs E of the user are included in the imaging ranges in the above-described state.

Thus, the arrangement example of the hardware of the information processing apparatus 10 according to the embodiment has been described above. While the example has been described above in which the information processing apparatus 10 according to the embodiment is implemented as a wearable device to be worn on the head of the user, the information processing apparatus 10 according to the embodiment is not limited to this example. The information processing apparatus 10 according to the embodiment may be a server, a general-purpose computer, a smartphone, a tablet, or the like that performs arithmetic processing based on a captured image. The information processing apparatus 10 according to the embodiment may be various apparatuses that perform arithmetic processing related to the line-of-sight estimation.

<<1.3. Functional Configuration Example of Information Processing Apparatus 10>>

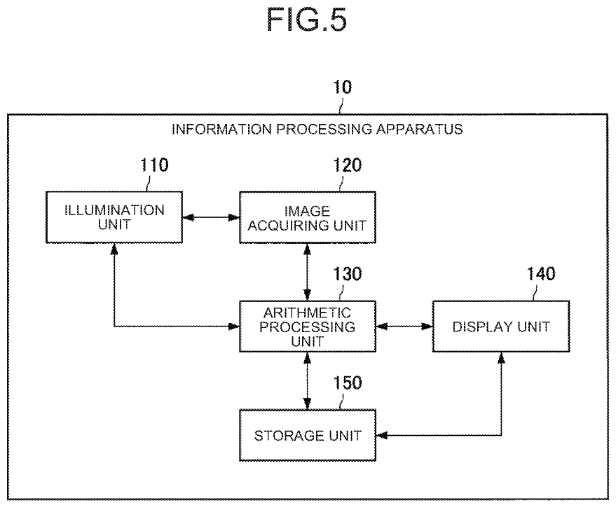

Next, a functional configuration example of the information processing apparatus 10 according to the embodiment will be described. FIG. 5 is a block diagram illustrating the functional configuration example of the information processing apparatus 10 according to the embodiment. With reference to FIG. 5, the information processing apparatus 10 according to the embodiment includes an illumination unit 110, an image acquiring unit 120, an arithmetic processing unit 130, a display unit 140, and a storage unit 150.

(Illumination Unit 110)

The illumination unit 110 according to the embodiment has a function to emit light to the eyeballs E of the user who has worn the information processing apparatus 10. To cope with this, the illumination unit 110 according to the embodiment includes the light source 103 that has been described above with reference to FIG. 3. The illumination unit 110 may emit light on the basis of control performed by the arithmetic processing unit 130.

(Image Acquiring Unit 120)

The image acquiring unit 120 according to the embodiment captures images of the eyeballs E of the user who has worn the information processing apparatus 10. More specifically, the image acquiring unit 120 acquires images of the eyeballs E including the bright points on the corneas of the user. To cope with this, the image acquiring unit 120 according to the embodiment includes the imaging unit 104 that has been described above with reference to FIG. 3. The image acquiring unit 120 may capture the images of the eyeballs E on the basis of control performed by the arithmetic processing unit 130.

(Arithmetic Processing Unit 130)

The arithmetic processing unit 130 according to the embodiment has a function to perform arithmetic processing related to the line-of-sight estimation for the user by using the three-dimensional eyeball model. Further, the arithmetic processing unit 130 may function as a control unit that controls each of the components included in the information processing apparatus 10. With the arithmetic processing unit 130 according to the embodiment, it is possible to realize the line-of-sight estimation with high accuracy by estimating an individual parameter related to the eyeball model for each of users. Meanwhile, the individual parameter according to the embodiment indicates an eyeball parameter that is unique to the user and that depends on a characteristic of an eyeball structure. The functions of the arithmetic processing unit 130 according to the embodiment will be described in detail later.

(Display Unit 140)

The display unit 140 according to the embodiment has a function to display visual information. The display unit 140 may display, for example, visual information corresponding to a user's line of sight that is estimated by the arithmetic processing unit 130. Further, the display unit 140 according to the embodiment displays a target point that is a target to be gazed at by the user, on the basis of control performed by the arithmetic processing unit 130. The display unit 140 according to the embodiment includes the display 102 that has been described above with reference to FIG. 3.

(Storage Unit 150)

The storage unit 150 according to the embodiment stores therein various kinds of information that are used for the line-of-sight estimation by the arithmetic processing unit 130. The storage unit 150 stores therein, for example, the eyeball parameters (individual parameters), such as the cornea-pupil distance and the corneal curvature radius, that are estimated by the arithmetic processing unit 130, various programs, calculation results, and the like.

Thus, the functional configuration of the information processing apparatus 10 according to the embodiment has been described above. The configuration as described above with reference to FIG. 5 is one example, and the functional configuration of the information processing apparatus 10 according to the embodiment is not limited to this example. For example, the information processing apparatus 10 according to the embodiment need not include the illumination unit 110, the image acquiring unit 120, the display unit 140, or the like. As described above, the information processing apparatus 10 according to the embodiment may be a server that performs arithmetic processing related to the line-of-sight estimation on the basis of an image that is captured by a different device, such as a wearable device. The functional configuration of the information processing apparatus 10 according to the embodiment may be flexibly deformed depending on specifications or operation.

<<1.4. Details of Line-of-Sight Estimation>>

Details of the line-of-sight estimation performed by the arithmetic processing unit 130 according to the embodiment will be described below. As has been described above with reference to FIG. 2, in the line-of-sight estimation using the corneal reflection method, an individual difference related to the eyeball parameters, such as the cornea-pupil distance and the corneal curvature radius, may become large factors to reduce the line-of-sight estimation accuracy.

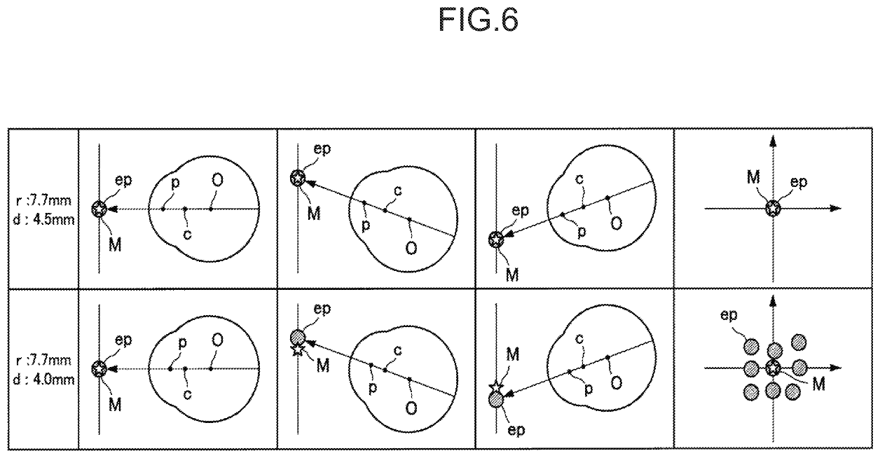

An example of reduction of the line-of-sight estimation accuracy due to the individual difference of the eyeball structure will be described below. FIG. 6 is a diagram illustrating an example of the reduction of the line-of-sight estimation accuracy due to the individual difference of the eyeball structure. In an upper part in FIG. 6, a positional relationship between a target point M and an estimated viewpoint position ep in a case where the eyeball structure of the user coincides with a general eyeball model when the line-of-sight estimation is performed using the general eyeball model. Further, in a lower part in FIG. 6, a positional relationship between the target point M and the estimated viewpoint position ep in a case where the eyeball structure of the user does not coincide with the general eyeball model when the line-of-sight estimation is performed using the general eyeball model. Meanwhile, in each of the upper part and the lower part in FIG. 6, a positional relationship obtained when the user keeps his/her gaze in a horizontal direction, a positional relationship obtained when the user keeps his/her gaze in an upward direction, a positional relationship obtained when the user keeps his/her gaze in a downward direction, and an error obtained by performing normalization using a target coordinate are illustrated from the left to the right.

Here, it is assumed that a corneal curvature radius r is 7.7 mm and a cornea-pupil distance d is 4.5 mm in the general eyeball model as described above. Further, the target point M is visual information that is displayed on the display unit 140 as a point to be gazed at by the user at the time of calibration. Meanwhile, for simplicity of explanation, FIG. 6 schematically illustrates a case in which it is assumed that there is no difference (deviation) between the optical axis and a visual axis.

When the upper part in FIG. 6 is focused on, in the case of the user who has the corneal curvature radius r and the cornea-pupil distance d that coincide with those of the general eyeball model, even if the user gazes at the target point M displayed in any direction, the estimated viewpoint position ep coincides with the target point M. In this manner, if the eyeball model coincides with the eyeball structure of the user, it is possible to realize the line-of-sight estimation with high accuracy.

In contrast, when the lower part in FIG. 6 is focused on, in the case of the user who has the cornea-pupil distance d (4.0 mm) different from that of the eyeball model, it is indicated that a large error occurs in the estimated viewpoint position ep when the user gazes at the target point M, such as an upper part or a lower part, for which an angular difference exists with respect to a front direction. In this manner, if the eyeball parameter, such as the cornea-pupil distance d or the corneal curvature radius r, related to the eyeball model is different from the eyeball structure of the user, an error between the target point M and the estimated viewpoint position ep increases and the line-of-sight estimation accuracy is extremely reduced.

To cope with this, the arithmetic processing unit 130 according to the embodiment realizes the line-of-sight estimation with high accuracy by dynamically estimating the eyeball parameter (individual parameter) unique to the user, for each user. In other words, the arithmetic processing unit 130 according to the embodiment is able to eliminate the factors for reduction, which has the largest influence on reduction of the line-of-sight estimation accuracy, by performing the line-of-sight estimation using a unique eyeball model that matches a characteristic of the eyeball structure of each of users.

Meanwhile, as one of features of the individual parameter estimated by the arithmetic processing unit 130 according to the embodiment, the individual parameter includes information on a relative position of a structure constituting the eyeball in a three-dimensional space. Here, the above-described structure includes two spherical structures and a pupil. Further, the two spherical structures include a cornea and an eyeball body including a corpus vitreum.

For example, the arithmetic processing unit 130 according to the embodiment may estimate, as the individual parameter, the cornea-pupil distance d that is a difference between the pupil center p and the corneal curvature center c that is a center of a cornea when the cornea is assumed as a spherical structure, and estimate a user's line of sight by using the cornea-pupil distance d.

Further, for example, if a vector connecting the corneal curvature center c and an eyeball center O that is a center of the eyeball body is used as an estimation result, the arithmetic processing unit 130 according to the embodiment may estimate, as the individual parameter, a distance between the corneal curvature center c and the eyeball center O.

Meanwhile, in the following, the explanation will be continued using the example in which the arithmetic processing unit 130 according to the embodiment estimates the cornea-pupil distance d as the individual parameter. The arithmetic processing unit 130 according to the embodiment is able to calculate an optical axis vector by estimating a position of the pupil center in a three-dimensional space by using the estimated cornea-pupil distance d.

In this case, the arithmetic processing unit 130 according to the embodiment may calculate the cornea-pupil distance d or the corneal curvature radius that minimizes an error between the target point M that is gazed at by the user and the visual axis or the optical axis. Here, the above-described error may be a distance or an angle between a vector that extends from the corneal curvature center c to the target point and the line-of-sight vector or the optical axis vector.

For example, the arithmetic processing unit 130 according to the embodiment calculates the cornea-pupil distance that minimizes the above-described error on the basis of a vector that extends from the corneal curvature center c to the target point M and a vector that extends from the corneal curvature center c to the pupil center p.

In this case, the arithmetic processing unit 130 according to the embodiment is able to estimate the cornea-pupil distance d and the corneal curvature radius r that are the individual parameters unique to the user, by formulating a problem with minimization of the error variance in the target coordinate system and obtaining a solution, for example.

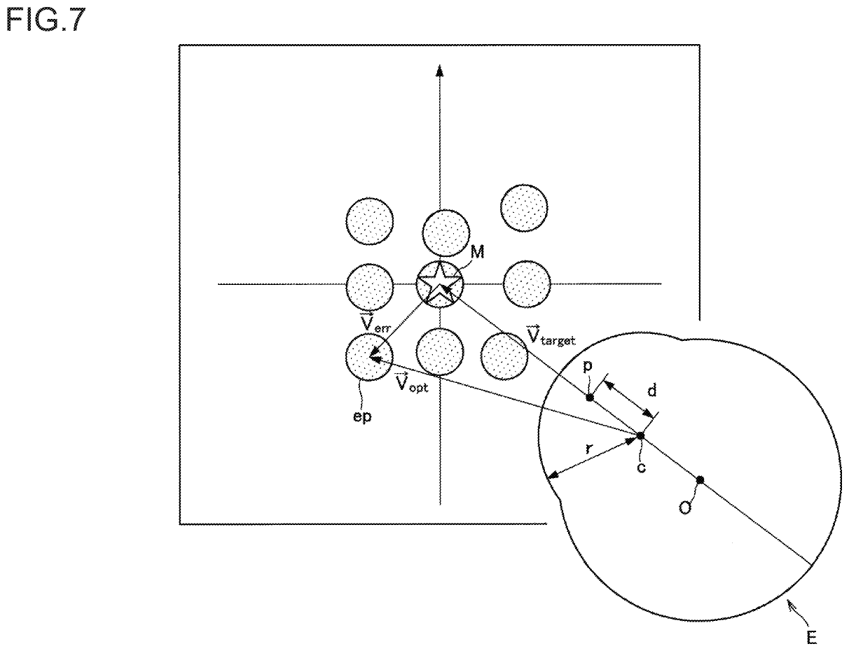

FIG. 7 is a diagram for explaining minimization of the error variance in the target coordinate system according to the embodiment. FIG. 7 illustrates a diagram in which an error between the target point M and the estimated viewpoint position ep is normalized in the target coordinate system.

In this case, the arithmetic processing unit 130 according to the embodiment obtains, by a full search method, a greedy algorithm, or the like, the corneal curvature radius r and the cornea-pupil distance d that minimize a vector Vₑᵣᵣ (added with a right arrow over V) that is a difference between a vector V_{target n} (added with a right arrow over V) that extends to the target point M gazed at by the user and a vector V_{opt n} (added with a right arrow over V) that extends to the estimated viewpoint position ep.

The arithmetic processing unit 130 according to the embodiment is able to obtain the corneal curvature radius r and the cornea-pupil distance d that minimize the error by, for example, Expression (1) below.

$${argmin}_{r , d} {\sum }_{a} {( {\vec{V}}_{err n} - \text{?} )}^{2} \\ {\tilde{V}}_{err n} \frac{{\tilde{V}}_{opt n}}{\vert {\tilde{V}}_{opt n} \vert} - \frac{{\tilde{V}}_{target n}}{\vert {\tilde{V}}_{target n} \vert} \\ \text{?} \text{indicates text missing or illegible when filed} \qquad ( 1 )$$

In this manner, according to the arithmetic processing unit 130 of the embodiment, by calculating the cornea-pupil distance d and the corneal curvature radius r for each of users, it is possible to generate the eyeball model that matches the eyeball structure of the user, and realize the line-of-sight estimation with high accuracy.

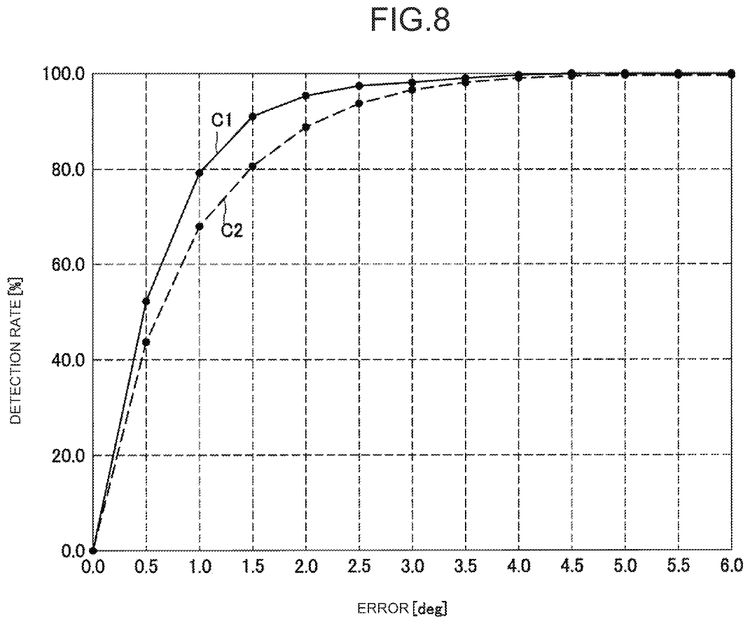

FIG. 8 is a diagram for explaining an effect of the line-of-sight estimation using the individual parameter that is estimated by the full search method according to the embodiment. A horizontal axis in FIG. 8 represents an error between a vector that extends to the target point M and the estimated line-of-sight vector, and a vertical axis in FIG. 8 represents a detection rate of each of errors.

Further, in FIG. 8, a solid segment C1 represents a result of the line-of-sight estimation in a case where the individual parameter is estimated by the full search method according to the embodiment, and a dashed segment C2 represents a result of the line-of-sight estimation in a case where the individual parameter is not estimated.

Here, for example, if the detection rates for an error of 1.5° are compared, it is indicated that improvement of 10% or more is achieved in the case where the individual parameter is estimated by the full search method according to the embodiment, as compared to the case where the individual parameter is not estimated.

In this manner, according to the arithmetic processing unit 130 of the embodiment, it is possible to realize the line-of-sight estimation with high accuracy by estimating the individual parameter, such as the cornea-pupil distance d or the corneal curvature radius r, for each of users and generating the eyeball model unique to the user.

Meanwhile, in the full search method as described above, input information for a plurality of target points M is needed to estimate the cornea-pupil distance d and the corneal curvature radius r. Therefore, when the individual parameter is estimated by the full search method, a process of presenting a target point to the user and capturing the eyeball image at the time the user gazes at the target point is repeated a plurality of number of times.

Further, in the full search method, an iterative calculation is performed to obtain the cornea-pupil distance d and the corneal curvature radius r that minimize the error, so that it takes a relatively long time to perform the arithmetic processing.

Therefore, the arithmetic processing unit 130 according to the embodiment may calculate the individual parameter, such as the cornea-pupil distance d, by a closed form that does not need an iterative calculation. With the above-descried function of the arithmetic processing unit 130 according to the embodiment, it becomes possible to analytically obtain the cornea-pupil distance d and dramatically increase the speed of the arithmetic processing.

Further, according to the closed-form solution of the embodiment, it is possible to calculate the cornea-pupil distance d by using only user's visual information on a single target point. In other words, the arithmetic processing unit 130 according to the embodiment is able to calculate the cornea-pupil distance d on the basis of a single eyeball image that is obtained when the user gazes at the target point. With the above-described function of the arithmetic processing unit 130 according to the embodiment, it becomes possible to largely simplify a process at the time of calibration and realize the line-of-sight estimation with high accuracy.

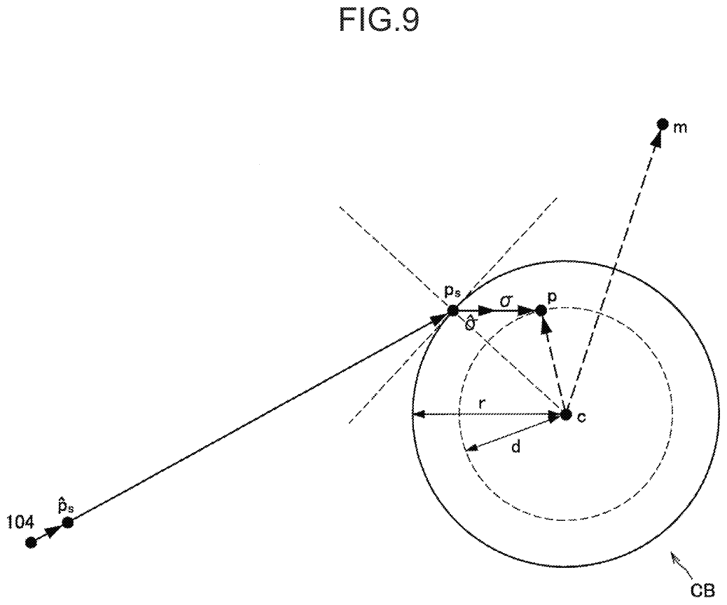

FIG. 9 is a diagram for explaining a calculation of the cornea-pupil distance d by the closed-form solution according to the embodiment. FIG. 9 illustrates a schematic diagram of a corneal sphere CB that is a spherical structure constituting the eyeball E of the user.

In the closed-form solution according to the embodiment, the arithmetic processing unit 130 is able to obtain the cornea-pupil distance d on the basis of input information on the target point that is given to the user at the time of calibration. Meanwhile, the above-described input information includes location information on the target point M, location information on the imaging unit 104, the eyeball image, and secondary information obtained from each piece of the information. Examples of the secondary information include information on a vector pₛ (hat symbol) that extends from an optical center of the imaging unit 104 to the pupil center pₛ on the corneal surface.

Further, it is assumed that the three-dimensional position of the corneal curvature center c and the corneal curvature radius r are already estimated. Meanwhile, it may be possible to use a method described in, for example, a literature “Beyond Alhazen's problem: Analytical Projection Model for Non-Central Catadioptric Cameras with Quadric Mirrors” (A Agrawal et al., 2011) to estimate the corneal curvature radius r.

Here, if a distance between pₛ and the pupil center p in the three-dimensional space is denoted by t, the pupil center p may be represented by Expression (2) below based on a known light refractive index. However, R in Expression (2) represents a set of real numbers. Further, a relationship between the distance t, which is between the pupil center pₛ on the corneal surface and the pupil center p in the three-dimensional space, and the cornea-pupil distance d may be represented by Expression (3) below.

p=pₛ+t\vec{σ} for t∈R (2)

∥p−c∥=d² (3)

Here, if Expression (2) is assigned to Expression (3) above, Expression is modified to Expression (4) below. Here, if T₁ and T₂ are defined by Expression (5) and Expression (6) below, the distance t between the pupil center pₛ on the corneal surface and the pupil center p in the three-dimensional space can be represented by a function of the cornea-pupil distance d as in Expression (7) below.

∥pₛ+t\hat{σ}−c∥=d²⇔t²+2(pₛ−c)·\vec{σ}t+∥pₛ−c∥²−d²=0 (4)

T₁=(pₛ−c)·\vec{σ} (5)

T₂=∥pₛ−c∥² (6)

t=−T₁±\sqrt{T₁²−T₂+d²} (7)

Here, an evaluation function L for calculating a square of a difference between 1.0 and an inner product of a unit vector that extends from the corneal curvature center c to the target point M and a unit vector that extends from the corneal curvature center c to the pupil center p is defined by Expression (8) below, and the cornea-pupil distance d that minimizes the evaluation function is obtained. In other words, the cornea-pupil distance d is defined by Expression (9) below.

$$\begin{matrix}L = \frac{( p - c )}{‖ p - c ‖} \cdot \frac{( m - c )}{‖ m - c ‖} - 1 = \frac{( p - c ) \cdot ( m - c )}{d ‖ m - c ‖} - 1 & ( 8 ) \\ d = {argmin}_{d} {L}^{2} & ( 9 )\end{matrix}$$

Subsequently, the evaluation function L represented by Expression (8) above is expanded and represented by a function of the above-described distance t and the cornea-pupil distance d. Specifically, if Expression (2) is assigned to Expression (8), it is possible to expand the evaluation function L as represented by Expression (10) below. Here, if K_{t,d}, K_{d}, K₁ are defined by Expression (11) below, the evaluation function L is represented by Expression (12) below as the function of the distance t and the cornea-pupil distance d.

$$\begin{matrix}L = \frac{1}{d ‖ m - c ‖} ( ( p , + t \dot{σ} - c ) \cdot ( m - c ) ) - 1 \\ = \frac{1}{d ‖ m - c ‖} ( t \hat{σ} \cdot ( m - c ) + ( p , - c ) \cdot ( m - c ) ) - 1 \\ = \frac{1}{‖ m - c ‖} ( \frac{t}{d} \hat{σ} \cdot ( m - c ) + \frac{1}{d} ( p , - c ) \cdot ( m - c ) - ‖ m - c ‖ ) & ( 10 ) \\ {K}_{t , d} = \hat{σ} \cdot ( m - c ) , \\ {K}_{d} = ( p , - c ) \cdot ( m - c ) , \\ {K}_{1} = - ‖ m - c ‖ & ( 11 ) \\ L = \frac{1}{‖ m - c ‖} ( {K}_{t , d} \frac{t}{d} + {K}_{d} \frac{1}{d} + {K}_{1} ) & ( 12 )\end{matrix}$$

Subsequently, the arithmetic processing unit 130 calculates the cornea-pupil distance d with which a derivative function of the evaluation function reaches zero. In this case, as represented by Expression (13), the arithmetic processing unit 130 first performs expansion to represent the derivative function of the evaluation function L by the cornea-pupil distance d. Further, if both sides of Expression (4) above are differentiated by the cornea-pupil distance d, Expression (14) below is obtained. Furthermore, if Expression (7) is assigned to Expression (14), it is possible to modify Expression (16) to Expression (17) below.

$$\begin{matrix}\frac{d L}{dd} = 0 \\ ⇔ \frac{d}{dd} ( {K}_{t , d} \frac{t}{d} + {K}_{d} \frac{1}{d} + {K}_{1} ) = 0 \\ ⇔ {K}_{t , d} \frac{d}{dd} ( \frac{t}{d} ) + {K}_{d} \frac{d}{dd} ( \frac{1}{d} ) = 0 \\ ⇔ {K}_{t , d} ( \frac{dt}{dd} \frac{1}{d} - \frac{t}{{d}^{2}} ) - {K}_{d} \frac{1}{{d}^{2}} = 0 & ( 13 ) \\ 2 t \frac{dt}{dd} + 2 {T}_{1} \frac{d L}{dd} - 2 d = 0 \\ ⇔ \frac{dt}{dd} ( t + {T}_{1} ) = d \\ ⇔ \frac{dt}{dd} = \frac{d}{t + {T}_{1}} & ( 14 ) \\ \frac{dt}{dd} = \pm \frac{d}{\sqrt{{T}_{1}^{2} - {T}_{2} + {d}^{2}}} & ( 15 )\end{matrix}$$

Subsequently, the arithmetic processing unit 130 assigns Expression (13) to Expression (15) above, and obtains the cornea-pupil distance d with which the derivative function of the evaluation function L reaches zero. In this case, the cornea-pupil distance d may be represented by Expression (16) below. However, T₁, T₂, K_{t,d}, K_{d}, K₁ in Expression (16) are defined by Expressions (5), (6), and (11) above.

$${K}_{t , d} ( \frac{dt}{dd} \frac{1}{d} - \frac{t}{{d}^{2}} ) - {K}_{d} \frac{1}{{d}^{2}} = 0 \\ ⇔ {K}_{t , d} ( \pm \frac{1}{\sqrt{{T}_{1}^{2} - {T}_{2} + {d}^{2}}} - \frac{- {T}_{1} \sqrt{{T}_{1}^{2} - {T}_{2} + {d}^{2}}}{{d}^{2}} ) - {K}_{d} \frac{1}{{d}^{2}} = 0 \\ ⇔ {K}_{t , d} ( {d}^{2} - {\sqrt{{T}_{1}^{2} - {T}_{2} + {d}^{2}}}^{2} ) \pm ( {K}_{t , d} {T}_{1} - {K}_{d} ) \sqrt{{T}_{1}^{2} - {T}_{2} + {d}^{2}} = 0 \\ ⇔ {K}_{t , d} ( {T}_{1}^{2} - {T}_{2} ) = \pm ( {K}_{t , d} {T}_{1} - {K}_{d} ) \sqrt{{T}_{1}^{2} - {T}_{2} + {d}^{2}} \\ ⇔ {{K}_{t , d}^{2} ( {T}_{1}^{2} - {T}_{2} )}^{2} = {( {K}_{t , d} {T}_{1} - {K}_{d} )}^{2} ( {T}_{1}^{2} - {T}_{2} + {d}^{2} ) \\ ⇔ \frac{{K}_{t , d}^{2}}{{( {K}_{t , d} {T}_{1} - {K}_{d} )}^{2}} {( {T}_{1}^{2} - {T}_{2} )}^{2} = {T}_{1}^{2} - {T}_{2} + {d}^{2} \\ ⇔ d = \pm \sqrt{\frac{{K}_{t , d}^{2}}{{( {K}_{t , d} {T}_{1} - {K}_{d} )}^{2}} {( {T}_{1}^{2} - {T}_{2} )}^{2} - ( {T}_{1}^{2} - {T}_{2} )} \qquad ( 16 )$$

In this manner, by obtaining the cornea-pupil distance d with which the derivative function of the evaluation function L reaches zero, the arithmetic processing unit 130 according to the embodiment is able to obtain the cornea-pupil distance d that minimizes the error, i.e., the cornea-pupil distance d corresponding to the eyeball structure of the user, without performing an iterative calculation.

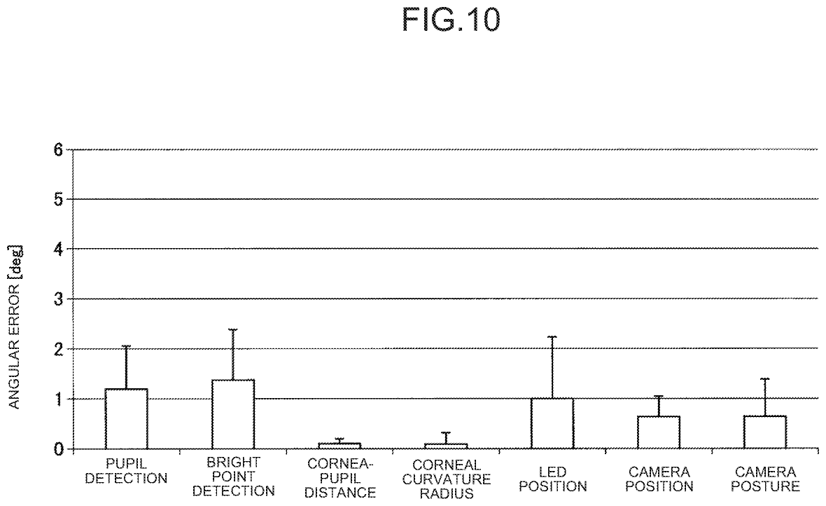

Next, effects realized by individual parameter estimation according to the embodiment will be described. FIG. 10 is a diagram for explaining improvement of the line-of-sight estimation accuracy by the individual parameter estimation according to the embodiment. FIG. 10 is a diagram illustrating a degree of influence of the factor to reduce the line-of-sight estimation accuracy in a case where the individual parameter estimation according to the embodiment and the calibration are performed. In FIG. 10, similarly to FIG. 2, a horizontal axis represents factors to reduce the line-of-sight estimation accuracy, and a vertical axis represents a magnitude of the angular error that occurs due to each of the factors.

Here, by comparison between FIG. 2 and FIG. 10, it is indicated that the angular error due to the cornea-pupil distance and the corneal curvature radius are largely reduced when the individual parameter estimation according to the embodiment is performed. Specifically, if the individual parameter estimation according to the embodiment is performed, the angular error due to cornea-pupil distance is reduced from about 3° to about 0.1°, and the angular error due to the corneal curvature radius is reduced from about 0.4° to about 0.1°.

In this manner, according to the method of estimating the individual parameter according to the embodiment, it is possible to largely improve the angular error due to the cornea-pupil distance that has the largest influence as the factor to reduce the line-of-sight estimation accuracy.

Further, Table 1 below is a table representing a measurement result of a processing time of the individual parameter estimation according to the embodiment. In this example, the individual parameter is estimated by combining estimation of the corneal curvature radius by a known method and the method of estimating the cornea-pupil distance according to the embodiment, and the processing time taken for the estimation is measured. Meanwhile, an error of the line-of-sight estimation due to each of combinations listed below does not largely vary.

TABLE 1

| | Estimation of | | |
|---|---|---|---|
| | Corneal Curvature | Method of Estimating | Processing |
| | Radius | Cornea-Pupil distance | Time [ms] |
| 1 | Not performed | Closed-form Solution | 0.20 |
| 2 | Performed | Closed-form Solution | 2.53 |
| 3 | Performed | Full Search Method | 23.62 |

First, if a case in which estimation of the corneal curvature radius and estimation of the cornea-pupil distance using the full search method according to the embodiment are performed and a case in which estimation of the corneal curvature radius and estimation of the cornea-pupil distance using the closed-form solution according to the embodiment are performed are compared, it is indicated that the processing speed is reduced to about one tenth in the case in which the closed-form solution is used, as compared to the case in which the full search method is used.

Further, if the estimation of the cornea-pupil distance using the closed-form solution according to the embodiment is performed and the estimation of the corneal curvature radius is not performed, it is possible to further increase the processing speed. Meanwhile, as described above, even if the estimation of the corneal curvature radius is not performed, the line-of-sight estimation accuracy is not reduced.

As described above, according to the individual parameter estimation performed by the arithmetic processing unit 130 of the embodiment, it is possible to perform the line-of-sight estimation using the eyeball model that matches the user, and it is possible to largely improve the accuracy of the line-of-sight estimation.

Further, according to the estimation of the cornea-pupil distance using the closed-form solution of the embodiment, it becomes possible to analytically obtain the cornea-pupil distance without performing an iterative calculation, and it is possible to improve the line-of-sight estimation accuracy and largely reduce the processing time.

<<1.5. Flow of Processes>>

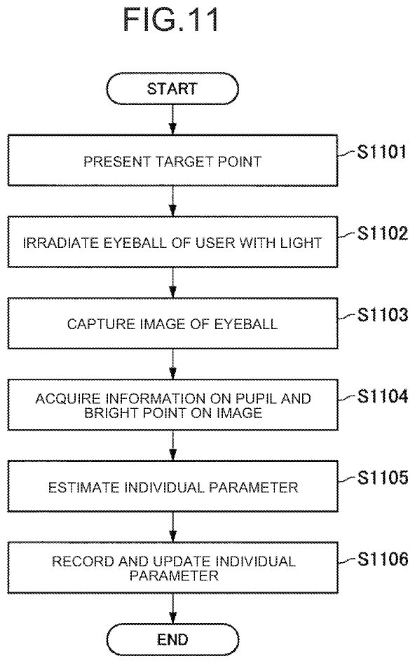

A flow of a line-of-sight estimation process performed by the information processing apparatus 10 according to the embodiment will be described below. FIG. 11 is a flowchart illustrating the flow of the line-of-sight estimation process according to the embodiment.

With reference to FIG. 11, first, the display unit 140 presents a target point as a gaze target to the user (S1101).

Subsequently, the illumination unit 110 irradiates the eyeball of the user with infrared light (S1102).

Then, the image acquiring unit 120 captures an image of the eyeball of the user who gazes at the target point displayed at Step S1101 (S1103).

Subsequently, the arithmetic processing unit 130 detects the pupil and the bright point from the eyeball image captured at Step S1203 and acquires the location information or the like (S1104).

Then, the arithmetic processing unit 130 performs an estimation process related to the individual parameter, such as the cornea-pupil distance or the corneal curvature radius (S1105).

Subsequently, the arithmetic processing unit 130 stores the individual parameter obtained at Step S1105 in the storage unit 150 (S1106).

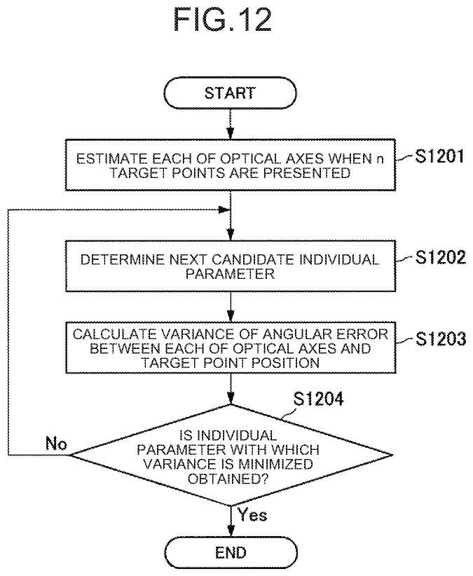

Next, details of the flow of the individual parameter estimation using a plurality of target points, such as the full search method or the greedy algorithm according to the embodiment, will be described. FIG. 12 is a flowchart illustrating a flow of the individual parameter estimation using the plurality of target points according to the embodiment.

With reference to FIG. 12, first, the arithmetic processing unit 130 estimates each of optical axes when n target points are presented (S1201).

Subsequently, the arithmetic processing unit 130 determines a next candidate individual parameter related to an optimum solution (S1202). Examples of the above-described individual parameter include the cornea-pupil distance and the corneal curvature radius.

Then, the arithmetic processing unit 130 calculates variance of an angular error between each of the optical axes and the target point (S1203).

Subsequently, the arithmetic processing unit 130 determines whether the individual parameter with which the variance of the angular error is minimized has been obtained (S1204).

Here, if the arithmetic processing unit 130 determines that the individual parameter by which the variance of the angular error is minimized has not yet been obtained (S1204: No), the arithmetic processing unit 130 returns to Step S1202 and repeats the subsequent processes.

In contrast, if the arithmetic processing unit 130 determines that the individual parameter by which the variance of the angular error is minimized has been obtained (S1204: Yes), the arithmetic processing unit 130 terminates the process related to the individual parameter estimation.

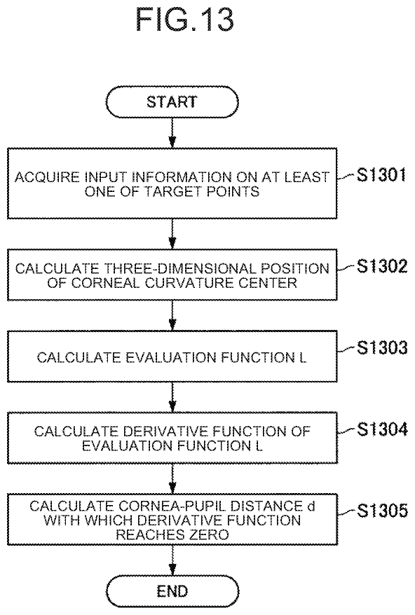

Next, a flow of the individual parameter estimation using the closed-form solution according to the embodiment will be described. FIG. 13 is a flowchart illustrating the flow of the individual parameter estimation using the closed-form solution according to the embodiment.

With reference to FIG. 13, first, the arithmetic processing unit 130 acquires input information on at least one of the target points (S1301).

Subsequently, the arithmetic processing unit 130 calculates a three-dimensional position of the corneal curvature center on the basis of the information acquired at Step S1301 (S1302).

Then, the arithmetic processing unit 130 performs the calculation related to the evaluation function L as described above (S1303).

Subsequently, the arithmetic processing unit 130 calculates the derivative function of the evaluation function L (S1304).

Then, the arithmetic processing unit 130 calculates the cornea-pupil distance d with which the derivative function calculated at Step S1304 reaches zero (S1305).

### 2. Hardware Configuration Example

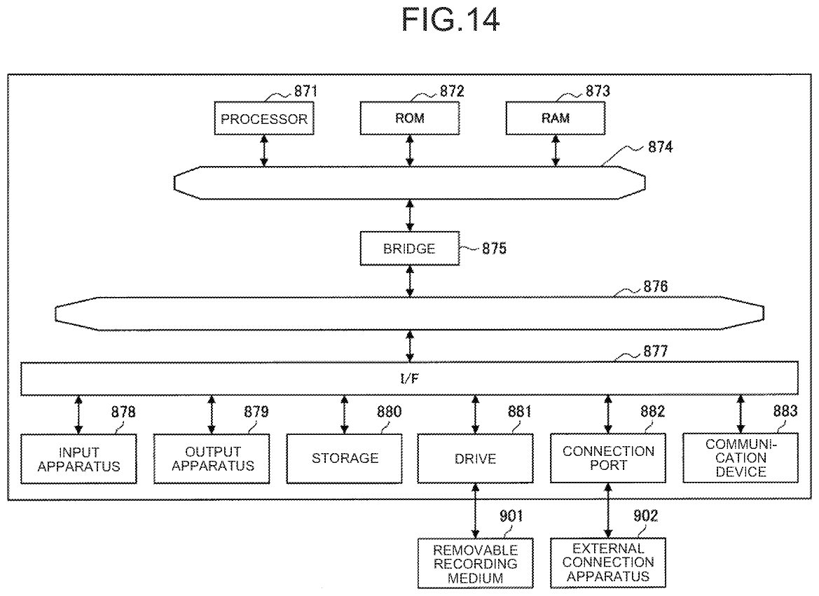

A hardware configuration example in a case where the information processing apparatus 10 according to one embodiment of the present disclosure is realized by a server or the like will be descried below. FIG. 14 is a block diagram illustrating an example of a hardware configuration of the information processing apparatus 10 according to one embodiment of the present disclosure. With reference to FIG. 14, the information processing apparatus 10 includes, for example, a processor 871, a read only memory (ROM) 872, a random access memory (RAM) 873, a host bus 874, a bridge 875, an external bus 876, an interface 877, an input apparatus 878, an output apparatus 879, a storage 880, a drive 881, a connection port 882, and a communication device 883. Meanwhile, the hardware configuration described here is one example, and part of the components may be omitted. Further, it may be possible to further include components other than the components described below.

(Processor 871)

The processor 871 functions as, for example, an arithmetic processing apparatus or a control apparatus, and controls whole or a part of operation of each of components on the basis of various programs stored in the ROM 872, the RAM 873, the storage 880, or a removable recording medium 901.

(ROM 872, RAM 873)

The ROM 872 is a means for storing a program read by the processor 871, data used for calculations, and the like. The RAM 873 temporarily or permanently stores therein, for example, a program read by the processor 871, various parameters that are appropriately changed when the program is executed, and the like.

(Host Bus 874, Bridge 875, External Bus 876, Interface 877)

The processor 871, the ROM 872, and the RAM 873 are connected to one another via the host bus 874 that is able to perform high-speed data transmission, for example. In contrast, the host bus 874 is connected to the external bus 876 for which a data transmission speed is relatively low, via the bridge 875, for example.

(Input apparatus 878)

As the input apparatus 878, for example, a mouse, a keyboard, a touch panel, a button, a switch, a lever, or the like may be used. Further, as the input apparatus 878, a remote controller (hereinafter, a remote) that is able to transmit a control signal using infrared or other radio waves may be used. Furthermore, the input apparatus 878 includes a voice input apparatus, such as a microphone.

(Output Apparatus 879)

The output apparatus 879 is an apparatus, such as a display apparatus including a cathode ray tube (CRT), a liquid crystal display (LCD), and an organic EL, an audio output apparatus including a speaker and a headphone, a printer, a cell-phone, or a facsimile machine, that is able to notify a user of the acquired information in a visual or auditory manner. Further, the output apparatus 879 according to the present disclosure includes various vibration devices that are able to output tactile stimulation.

(Storage 880)

The storage 880 is an apparatus for storing various kinds of data. As the storage 880, for example, a magnetic storage device, such as a hard disk drive (HDD), a semiconductor storage device, an optical storage device, or a magneto optical disk may be used.

(Drive 881)

The drive 881 is an apparatus that reads information stored in the removable recording medium 901, such as a magnetic disk, an optical disk, a magneto optical disk, or a semiconductor memory, or writes information in the removable recording medium 901.

(Removable Recording Medium 901)

The removable recording medium 901 is, for example, a digital versatile disk (DVD) medium, a Blu-ray (registered trademark) medium, an HD DVD medium, or various kinds of semiconductor storage media. The removable recording medium 901 may of course be an integrated circuit (IC) card equipped with a contactless IC chip, an electronic device, or the like.

(Connection Port 882)

The connection port 882 is, for example, a port, such as a universal serial bus (USB) port, an IEEE1394 port, a small computer system interface (SCSI), an RS-232C port, or an optical audio terminal, for connecting an external connection apparatus 902.

(External Connection Apparatus 902)

The external connection apparatus 902 is, for example, a printer, a portable music player, a digital camera, a digital video camera, an IC recorder, or the like.

(Communication Device 883)

The communication device 883 is a communication device for connection to a network, and is, for example, a communication card for a wired or wireless LAN, Bluetooth (registered trademark), or a wireless USB (WUSB), a router for optical communication, a router for asymmetric digital subscriber line (ADSL), a modem for various kinds of communication, or the like.

### 3. Conclusion

As described above, the information processing apparatus 10 according to one embodiment of the present disclosure includes the arithmetic processing unit 130 that performs arithmetic processing related to estimation of a user's line of sight by using the eyeball model. Further, the arithmetic processing unit 130 according to one embodiment of the present disclosure has a feature to dynamically estimate the individual parameter related to the eyeball model for each of users. Furthermore, the above-described individual parameter includes information on a relative position of a structure constituting the eyeball in a three-dimensional space. With this configuration, it is possible to realize the line-of-sight estimation with high accuracy in accordance with an individual characteristic.

Thus, preferred embodiments of the present disclosure have been described in detail above with reference to the accompanying drawings, but a technical scope of the present disclosure is not limited to this example. A person skilled in the art may conceive various alternations and modifications within the scope of the technical ideas of the appended claims, and it should be understood that they will naturally come under the technical scope of the present disclosure.

For example, in the embodiment as described above, a case has been described as a main example in which the information processing apparatus 10 performs the line-of-sight estimation using the corneal reflection method, but a technical idea according to the present disclosure is widely applicable to various apparatuses, such as an iris authentication apparatus and a surgical pupil tracking apparatus, that use a three-dimensional eyeball model.

Further, the effects described in this specification are merely illustrative or exemplified effects, and are not limitative. That is, with or in the place of the above effects, the technology according to the present disclosure may achieve other effects that are clear to those skilled in the art from the description of this specification.

Furthermore, it may be possible to generate a program that causes hardware, such as a CPU, a ROM, and a RAM, incorporated in a computer to exert the same functions as those of the information processing apparatus 10, and it may be possible to provide a computer-readable recording medium in which the program is recorded.

Moreover, each of Steps in the processes performed by the information processing apparatus 10 of the present specification need not always be processed in chronological order as illustrated in the flowchart. For example, each of Steps related to the processes performed by the information processing apparatus 10 may be executed in different order from the order illustrated in the flowchart, or may be performed in a parallel manner.

Furthermore, Expressions described in the present specification are one example, and the information processing apparatus 10 according to one embodiment of the present disclosure need not always perform processes using Expressions as described above. Moreover, it should be understood that the modified Expressions will come under the technical scope of the present disclosure.

Additionally, the following configurations are also within the technical scope of the present disclosure.

(1)

An information processing apparatus comprising:

an arithmetic processing unit that performs arithmetic processing related to estimation of a line of sight of a user by using an eyeball model, wherein

the arithmetic processing unit dynamically estimates an individual parameter related to the eyeball model for each of users, and

the individual parameter includes information on a relative position of a structure constituting an eyeball in a three-dimensional space.

(2)

The information processing apparatus according to (1), wherein the structure includes two spherical structures and a pupil.

(3)

The information processing apparatus according to (1) or (2), wherein

the individual parameter includes a cornea-pupil distance that is a distance between a pupil center and a corneal curvature center, and

the arithmetic processing unit estimates the line of sight of the user by using the cornea-pupil distance.

(4)

The information processing apparatus according to (3), wherein the arithmetic processing unit estimates a position of the pupil center in a three-dimensional space by using the cornea-pupil distance.

(5)

The information processing apparatus according to (3) or (4), wherein the arithmetic processing unit calculates the cornea-pupil distance with which an error between a target point that is gazed at by the user and at least one of a visual axis and an optical axis is minimized.

(6)

The information processing apparatus according to (5), wherein the error includes one of a distance and an angle between a vector that extends from a corneal curvature center to the target point and at least one of a line-of-sight vector and an optical axis vector.

(7)

The information processing apparatus according to (5) or (6), wherein the arithmetic processing unit calculates the cornea-pupil distance with which the error is minimized, on the basis of a vector that extends from the corneal curvature center to the target point and a vector that extends from the corneal curvature center to the pupil center.

(8)

The information processing apparatus according to any one of (5) to (7), wherein the arithmetic processing unit calculates the cornea-pupil distance on the basis of input information on the single target point.

(9)

The information processing apparatus according to any one of (5) to (8), wherein the arithmetic processing unit calculates the cornea-pupil distance on the basis of a single eyeball image that is obtained when the user gazes at the target point.

(10)

The information processing apparatus according to any one of (5) to (8), wherein the arithmetic processing unit calculates the cornea-pupil distance by using a closed form.

(11)

The information processing apparatus according to any one of (5) to (8), wherein the arithmetic processing unit calculates the cornea-pupil distance by using an evaluation function that minimizes the error.

(12)

The information processing apparatus according to (11), wherein the arithmetic processing unit calculates the cornea-pupil distance by differential operation on the evaluation function.

(13)

The information processing apparatus according to (12), wherein the arithmetic processing unit calculates the cornea-pupil distance with which a derivative function of the evaluation function reaches zero.

(14)

The information processing apparatus according to any one of (1) to (13), wherein

the individual parameter includes a corneal curvature radius, and

the arithmetic processing unit estimates the line of sight of the user by using the estimated corneal curvature radius.

(15)

The information processing apparatus according to any one of (1) to (14), wherein the arithmetic processing unit estimates the line of sight of the user by a corneal reflection method.

(16)

The information processing apparatus according to any one of (1) to (15), further comprising:

an image acquiring unit that acquires an image including a bright point on a cornea of the user.

(17)

The information processing apparatus according to any one of (5) to (13), further comprising:

a display unit that displays the target point.

(18)

The information processing apparatus according to any one of (1) to (17), wherein the information processing apparatus is a device that is worn on a head of the user.

(19)

An information processing method comprising:

performing, by a processor, arithmetic processing related to estimation of a line of sight of a user by using an eyeball model, wherein

performing the arithmetic processing includes dynamically estimating an individual parameter related to the eyeball model for each of users, and

the individual parameter includes information on a relative position of a structure constituting an eyeball in a three-dimensional space.

(20)

A program that causes a computer to function as an information processing apparatus that includes:

an arithmetic processing unit that performs arithmetic processing related to estimation of a line of sight of a user by using an eyeball model, wherein

the arithmetic processing unit dynamically estimates an individual parameter related to the eyeball model for each of users, and

the individual parameter includes information on a relative position of a structure constituting an eyeball in a three-dimensional space.

### REFERENCE SIGNS LIST

10 Information processing apparatus
110 Illumination unit
120 Image acquiring unit
130 Arithmetic processing unit
140 Display unit
150 Storage unit
c Corneal curvature center
p Pupil center
d Cornea-pupil distance
r Corneal curvature radius

1. An information processing apparatus comprising:
an arithmetic processing unit that performs arithmetic processing related to estimation of a line of sight of a user by using an eyeball model, wherein
the arithmetic processing unit dynamically estimates an individual parameter related to the eyeball model for each of users, and
the individual parameter includes information on a relative position of a structure constituting an eyeball in a three-dimensional space.

2. The information processing apparatus according to claim 1, wherein the structure includes two spherical structures and a pupil.

3. The information processing apparatus according to claim 1, wherein
the individual parameter includes a cornea-pupil distance that is a distance between a pupil center and a corneal curvature center, and
the arithmetic processing unit estimates the line of sight of the user by using the cornea-pupil distance.

4. The information processing apparatus according to claim 3, wherein the arithmetic processing unit estimates a position of the pupil center in a three-dimensional space by using the cornea-pupil distance.

5. The information processing apparatus according to claim 3, wherein the arithmetic processing unit calculates the cornea-pupil distance with which an error between a target point that is gazed at by the user and at least one of a visual axis and an optical axis is minimized.

6. The information processing apparatus according to claim 5, wherein the error includes one of a distance and an angle between a vector that extends from a corneal curvature center to the target point and at least one of a line-of-sight vector and an optical axis vector.

7. The information processing apparatus according to claim 5, wherein the arithmetic processing unit calculates the cornea-pupil distance with which the error is minimized, on the basis of a vector that extends from the corneal curvature center to the target point and a vector that extends from the corneal curvature center to the pupil center.

8. The information processing apparatus according to claim 5, wherein the arithmetic processing unit calculates the cornea-pupil distance on the basis of input information on the single target point.

9. The information processing apparatus according to claim 5, wherein the arithmetic processing unit calculates the cornea-pupil distance on the basis of a single eyeball image that is obtained when the user gazes at the target point.

10. The information processing apparatus according to claim 5, wherein the arithmetic processing unit calculates the cornea-pupil distance by using a closed form.

11. The information processing apparatus according to claim 5, wherein the arithmetic processing unit calculates the cornea-pupil distance by using an evaluation function that minimizes the error.

12. The information processing apparatus according to claim 11, wherein the arithmetic processing unit calculates the cornea-pupil distance by differential operation on the evaluation function.

13. The information processing apparatus according to claim 12, wherein the arithmetic processing unit calculates the cornea-pupil distance with which a derivative function of the evaluation function reaches zero.

14. The information processing apparatus according to claim 1, wherein
the individual parameter includes a corneal curvature radius, and
the arithmetic processing unit estimates the line of sight of the user by using the estimated corneal curvature radius.

15. The information processing apparatus according to claim 1, wherein the arithmetic processing unit estimates the line of sight of the user by a corneal reflection method.

16. The information processing apparatus according to claim 1, further comprising:
an image acquiring unit that acquires an image including a bright point on a cornea of the user.

17. The information processing apparatus according to claim 5, further comprising:
a display unit that displays the target point.

18. The information processing apparatus according to claim 1, wherein the information processing apparatus is a device that is worn on a head of the user.

19. An information processing method comprising:
performing, by a processor, arithmetic processing related to estimation of a line of sight of a user by using an eyeball model, wherein
performing the arithmetic processing includes dynamically estimating an individual parameter related to the eyeball model for each of users, and
the individual parameter includes information on a relative position of a structure constituting an eyeball in a three-dimensional space.

20. A program that causes a computer to function as an information processing apparatus that includes:
an arithmetic processing unit that performs arithmetic processing related to estimation of a line of sight of a user by using an eyeball model, wherein
the arithmetic processing unit dynamically estimates an individual parameter related to the eyeball model for each of users, and
the individual parameter includes information on a relative position of a structure constituting an eyeball in a three-dimensional space.
